SEQUENCE LISTING (4 sequences)

SEQ ID NO: 1 - DNA, 20 bp, Artificial Sequence
  Description of Artificial Sequence Synthetic adapter sequence
gatcggaagagcacacgtct

SEQ ID NO: 2 - DNA, 33 bp, Artificial Sequence
  Description of Artificial Sequence Synthetic adapter sequence
acactctttccctacacgacgctcttccgatct

SEQ ID NO: 3 - DNA, 58 bp, Artificial Sequence
  Description of Artificial Sequence Synthetic adapter sequence
aatgatacggcgaccaccgagatctacactctttccctacacgacgctcttccgatct

SEQ ID NO: 4 - DNA, 64 bp, Artificial Sequence
  Description of Artificial Sequence Synthetic adapter sequence
caagcagaagacggcatacgagatcgtgatgtgactggagttcagacgtgtgctcttccgatct

(19) United States
(12) Patent Application Publication
(10) Pub. No.: US 2018/0320169 A1
(43) Pub. Date: Nov. 8, 2018

(54) METHOD FOR CONSTRUCTING DNA LIBRARY FOR SEQUENCING

(71) Applicants: NANJING ANNOROAD GENE TECHNOLOGY CO. LTD, Nanjing (CN); ANNOROAD GENE TECHNOLOGY (BEIJING) CO., LTD, Beijing (CN)

(72) Inventors: Chongjian CHEN, Beijing (CN); Yun XIA, Beijing (CN); Qiongfang PENG, Beijing (CN); Zhandong WANG, Beijing (CN); Yuehan XUE, Beijing (CN); Zhaoling XUAN, Beijing (CN); Dawei LI, Beijing (CN); Junbin LIANG, Beijing (CN)

(73) Assignees: NANJING ANNOROAD GENE TECHNOLOGY CO. LTD, Nanjing (CN); ANNOROAD GENE TECHNOLOGY (BEIJING) CO., LTD, Beijing (CN)

(21) Appl. No.: 15/776,106

(22) PCT Filed: Nov. 17, 2015
(86) PCT No.: PCT/CN2015/094786

§ 371 Date: May 15, 2018

(51) Int. Cl. C12N 15/10; C12Q 1/6874

(52) U.S. Cl. CPC C12N 15/1068; C12Q 1/6874

(57) ABSTRACT

Provided is a method for constructing a DNA library for sequencing. The method comprises A-tailing of a single 3′ end to at least a portion of blunt-end DNA fragments, and a step of blunt-end ligating the obtained DNA fragments having A tailing to the single 3′ end. Also provided are a DNA library for sequencing constructed with the method and a corresponding sequencing method.

### FIELD OF THE INVENTION

The present invention pertains to the field of molecular biology, and in particular, relates to a method for constructing a DNA library for sequencing, a DNA library for sequencing constructed by the method, and the application of the DNA library for sequencing in sequencing.

### BACKGROUND OF THE INVENTION

In general, second-generation DNA sequencing is performed using a DNA library for sequencing containing double-stranded DNA for sequencing. In the double-stranded DNA for sequencing, a single DNA fragment to be sequenced is inserted between a 5′ sequence and a 3′ sequence, and the 5′ sequence and the 3′ sequence respectively contain a base sequence matched with a sequencing primer. Due to the limitations of the second-generation DNA sequencing technology, the length of the single DNA fragment to be sequenced is usually about 10 to 500 bp.

In the second-generation DNA sequencing using the aforementioned DNA library for sequencing, it is necessary to read data separately for each double-stranded DNA for sequencing which contains a single DNA fragment to be sequenced, that is, the data of only one DNA fragment to be sequenced can be obtained in one sequencing process, although the existing sequencers can already read data from both ends of the double-stranded DNA simultaneously. When this conventional DNA library for sequencing is used for sequencing, the dosage of sequencing reagents is large and the time for sequencing is long, resulting in a high cost and a low efficiency of sequencing.

### SUMMARY OF THE INVENTION

In view of the above deficiencies existing in the prior art, an object of the present invention is to provide a method for constructing a DNA library for sequencing and a sequencing method using a DNA library for sequencing constructed using the method for constructing a DNA library for sequencing, wherein, in the sequencing using a DNA library for sequencing constructed using the method for constructing a DNA library for sequencing, the dosage of sequencing reagents can be reduced and the time for sequencing can be shortened, thereby reducing the sequencing cost and improving the sequencing efficiency.

The inventors of the present invention conducted intensive studies in order to solve the above technical problems, and as a result, they have found that by changing the step of A-tailing of double 3′ ends into a step of A-tailing of a single 3′ end and adding a step of blunt-end ligation in the conventional method for constructing a DNA library for sequencing, it is possible to insert two or more DNA fragments to be sequenced between the 5′ sequence and the 3′ sequence of the double-stranded DNA for sequencing, which makes it possible to simultaneously read the data of two DNA fragments to be sequenced from both ends of the double-stranded DNA in the sequencing step, thereby reducing the dosage of sequencing reagents and shortening the time for sequencing.

That is, the present invention based on the findings aforementioned includes:

1. A method for constructing a DNA library for sequencing, comprising:

step B-1: A-tailing of a single 3′ end to at least a portion of a blunt-end DNA fragment to obtain a DNA fragment having A tailing to the single 3′ end; and

step B-2: blunt-end ligating the DNA fragments with an A-tail at the single 3′ end obtained in step B-1.

2. The method for constructing a DNA library for sequencing according to item 1, further comprising:

step A performed before the step B: end-repairing DNA fragments to be sequenced to obtain blunt-end DNA fragments;

step C performed after the step B-2 or simultaneously with the step B-2: adding adapters to the blunt-end ligated products of the step B-2 to obtain adapter-added DNA fragments; and

step D performed after the step C: subjecting the adapter-added DNA fragments to PCR amplification to obtain amplification products.

3. The method for constructing a DNA library for sequencing according to item 1 or 2, further comprising:

step E performed after the step D: fragment selection of the amplification products.

4. The method for constructing a DNA library for sequencing according to any one of items 1 to 3, further comprising:

step F performed after the step E: purifying the amplification products after the fragment selection.

5. The method for constructing a DNA library for sequencing according to any one of items 1 to 4, further comprising:

step C1 performed after the step C: fragment selection of the adapter-added DNA fragments; and in the step D, subjecting the adapter-added DNA fragments after the fragment selection to PCR amplification to obtain amplification products.

6. The method for constructing a DNA library for sequencing according to any one of items 1 to 5, wherein the amplification products containing two or more DNA fragments to be sequenced is selected.

7. A DNA library for sequencing, wherein double-stranded DNAs containing two or more DNA fragments to be sequenced makes up at least 10 mol % of the total double-stranded DNAs.

8. The DNA library for sequencing according to item 7, which is constructed by the method for constructing a DNA library for sequencing according to any one of items 1 to 6.

9. A sequencing method, wherein sequencing is conducted taking the DNA library for sequencing according to item 7 or 8 as an object.

10. The sequencing method according to item 9, wherein the sequencing is double-stranded sequencing.

11. The sequencing method according to item 9 or 10, wherein the sequencing is conducted using Illumina platform.

### EFFECT OF THE INVENTION

In the double-stranded DNAs for sequencing contained in the DNA library for sequencing constructed using the method for constructing a DNA library for sequencing, two or more DNA fragments to be sequenced are allowed to be inserted between the 5′ sequence and the 3′ sequence, which makes it possible to read data from both ends of the double-stranded DNAs for sequencing simultaneously in one sequencing process in case of using a paired-end sequencing mode, and thus the dosage of sequencing reagents can be reduced and the time for sequencing can be shortened, thereby reducing the sequencing cost and improving the sequencing efficiency. In addition, the technology of A-tailing of a single 3′ end saves the reagents used to construct libraries and reduces the cost of constructing DNA libraries and sequencing.

### DESCRIPTION OF DRAWINGS

FIG. 1 is a comparison diagram between the library construction process of the present invention and a conventional library construction process;

FIG. 2 is a schematic diagram of results of DNA library fragments sizes testing using Agilent 2100 Bioanalyzer of Example 2;

FIG. 3 is a diagram of a double-end sequencing base distribution of Example 3;

FIG. 4 is a diagram of a single-end sequencing base distribution of Example 3;

FIG. 5 is a diagram of a double-end sequencing mass distribution of Example 3;

FIG. 6 is a diagram of a single-end sequencing mass distribution of Example 3.

### DETAILED DESCRIPTION OF THE INVENTION

First, in one aspect, the present invention provides a method for constructing a DNA library for sequencing (the method for constructing a DNA library for sequencing of the present invention), comprising:

step B-1: A-tailing of a single 3′ end for a blunt-end DNA fragment to obtain a DNA fragment having A tailing to the single 3′ end; and

step B-2: blunt-end ligating the DNA fragment having A tailing to the single 3′ end obtained in step B-1.

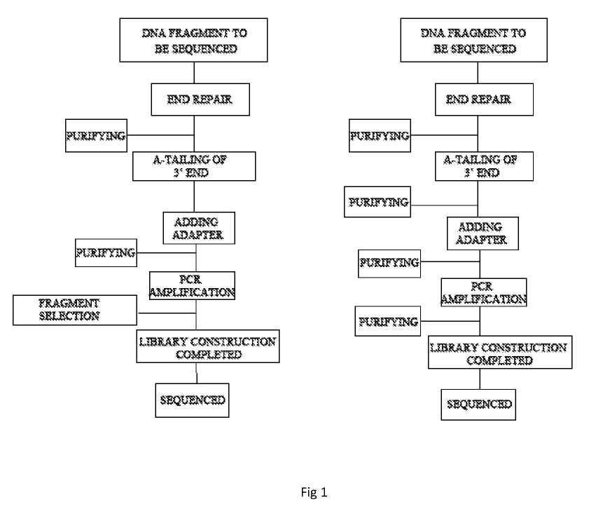

As shown in the right diagram of FIG. 1, in contrast to the conventional method for constructing a DNA library for sequencing where blunt-end DNA fragments obtained by end repair is added with A bases to double 3′ ends, in the method for constructing a DNA library for sequencing of the present invention, blunt-end DNA fragments obtained by end repair is subjected to A-tailing of a single 3′ end to obtain DNA fragments having A tailing to a single 3′ end, and moreover, the obtained DNA fragments with A tailing to a single 3′ end are blunt-end ligated in the method for constructing a DNA library for sequencing of the present invention. Through the above steps, the DNA fragments containing two or more DNA fragments to be sequenced obtained by blunt-end ligation can be inserted as a whole between the 5′ sequence and the 3′ sequence of the double-stranded DNA for sequencing in a subsequent step.

The A-tailing of a single 3′ end of the blunt-end DNA fragment described above can be achieved by any method known to the skilled person in the art, for example, by using DNA polymerases having the above function (e.g., Klenow fragment lacking 3′ end to 5′ end exonuclease activity). The skilled person in the art can easily achieve A-tailing of a single 3′ end by reducing the dosage of enzyme as used, lowering the reaction temperature, and/or shortening the reaction time based on the conventional enzyme reaction of A-tailing of double 3′ ends.

The blunt-end ligation of the DNA fragments having A tailing to a single 3′ end described above can be achieved by any method known to the skilled person in the art, for example, by using DNA ligases with a blunt end ligation function (e.g., T4 DNA ligase, T3 DNA ligase).

Preferably, the method for constructing a DNA library for sequencing of the present invention further comprises:

step A performed before the step B: end-repairing DNA fragments to be sequenced to obtain blunt-end DNA fragments;

step C performed after the step B-2 or simultaneously with the step B-2: adding adapters to the blunt-end ligated products of the step B-2 to obtain adapter-added DNA fragments; and

step D performed after the step C: subjecting the adapter-added DNA fragments to PCR amplification to obtain amplification products.

The above steps A, C and D can be performed by conventional methods in the technical field. It should be noted that since step B-2 and step C can be performed by using some DNA ligases (e.g., T4 DNA ligase, T3 DNA ligase), step B-2 and step C can be performed simultaneously by using the aforementioned DNA ligases (e.g., T4 DNA ligase, T3 DNA ligase).

The “DNA fragments to be sequenced” in step A is not particularly limited and is preferably DNA fragments about 10 to 1000 bp, more preferably about 20 to 800 bp, more preferably about 30 to 750 bp, more preferably about 40 to 700 bp, more preferably about 50 to 650 bp, more preferably about 100 to 600 bp, more preferably about 150 to 550 bp, from a sequencer acceptance perspective.

Preferably, the method for constructing a DNA library for sequencing of the present invention further comprises step E performed after the step D: fragment selection of the amplification products.

Preferably, step C1 performed after the step C: fragment selection of the adapter-added DNA fragments; and in the step D, the adapter-added DNA fragments after the fragment selection is subjected to PCR amplification to obtain amplification products.

In the step C1 and the step E, it is preferable to select the amplification products in which two or more DNA fragments to be sequenced are inserted (that is, to remove as much as possible amplification products in which only one DNA fragment to be sequenced is inserted, or amplification products in which no DNA fragment to be sequenced is inserted).

The fragment selection described above may be performed by a conventional method in the technical field, for example, by using magnetic beads. In the case of using magnetic beads for fragment selection, the dosage of the magnetic beads can be adjusted according to the size of the amplification products to be selected, for example, so as to obtain the specific amplification products.

Preferably, a step of purifying DNA fragments may be added between each step of the method for constructing a DNA library for sequencing of the present invention, for example, between step A and step B-1, between step B-1 and step C, between step C and step C1, between step C (or C1) and step D, between step D and step E, and/or after step E.

This purification step can be carried out by using a conventional method in the technical field, for example, by using purified magnetic beads.

The method for constructing a DNA library for sequencing of the present invention is significantly different from the conventional method in that it seeks to integrate two or more DNA fragments to be sequenced in a double-stranded DNA for sequencing, while the conventional method avoids such situation.

Therefore, in one aspect, the present invention provides a DNA library for sequencing (the DNA library for sequencing of the present invention), wherein the ratio of the double-stranded DNAs containing two or more DNA fragments to be sequenced to the total double-stranded DNAs is notably higher than that in a conventional DNA library for sequencing. This ratio may be, for example, at least 10 mol %, preferably at least 20 mol %, more preferably at least 30 mol %, more preferably at least 40 mol %, more preferably at least 50 mol %, more preferably at least 60 mol %, more preferably at least 70 mol %, more preferably at least 80 mol %, more preferably at least 90 mol %.

The DNA library for sequencing of the present invention may be constnicted by using the method for constructing a DNA library for sequencing of the present invention, for example.

In addition, in one aspect, the present invention provides a sequencing method (the sequencing method of the present invention), wherein sequencing is conducted taking the DNA library for sequencing of the present invention as an object.

In addition to taking the DNA library for sequencing of the present invention as an object, the sequencing method of the present invention may be conducted using a conventional method in the technical field. Preferably, the sequencing method of the present invention may employ a paired-end sequencing, for example, a paired-end sequencing using Illumina platform (e.g., HiSeq 2500 or NextSeq 500).

### EXAMPLES

The present invention will be described more specifically below with examples. It should be understood that the examples described herein are intended to explain the present invention, rather than to limit the present invention.

### Example 1

### Constructing a DNA Library for Sequencing using the Method of the Present Invention

(1) Preparation of DNA fragments to be sequenced

The DNA fragments to be sequenced used in the present example are a mixture of some DNA fragments about 300 bp and some DNA fragments about 180 bp. Some of the double-stranded DNA fragments are blunt-ended, and the others contain 3′ or 5′ sticky end.

(2) End repairing to obtain blunt-end DNA fragments

A. The reaction system is as follows:

| Reagent | Volume |
|---|---|
| DNA fragments obtained in step (1) | 42 μL |
| Mixture of dNTPs (10 mM) | 1 μL |
| T4 DNA polymerase | 1 μL |
| T4 PNK (T4 polynucleotide kinase) | 1 μL |
| PNK buffer | 5 μL |
| In total | 50 μL |

B. Incubating in a PCR instrument for 30 minutes at a temperature of 20° C.;

C. Purifying the reaction product by magnetic beads, eluting with 19.5 μL of elution buffer EB (Elution Buffer) to obtain blunt-end DNA fragments.

(3) A-tailing of a single 3′ end to blunt-end DNA fragments

The reaction system is as follows:

| Reagent | Volume |
|---|---|
| DNA fragments obtained in step (2) | 19.5 μL |
| Klenow buffer (10×) | 2.5 μL |
| dATP (1 mM) | 2.5 μL |
| Klenow fragments (lacking 3′ to 5′ exonuclease activity) | 0.05 μL |
| ddH₂O | 0.45 μL |
| In total | 25 μL |

They were incubated for 30 minutes in a PCR instrument at a temperature of 37° C.

(4) Specific adapter ligation and purification;

In order to perform specific amplification in PCR, it is necessary to add a specific adapter to both ends of the fragments having A tailing to the single 3′ end obtained in (3), and the adapter sequences required for the ligation reaction need to be mixed in equal proportion when in use. The adapter sequences are as follows:

Adapter sequence 1:
5′ P-GATCGGAAGAGCACACGTCT-3′
Adapter sequence 2:
5′ P-ACACTCTTTCCCTACACGACGCTCTTCCGATCT-3′

A. The reaction system is as follows:

| Reagent | Volume |
|---|---|
| DNA fragments obtained in step (3) | 25 μL |
| DNA ligase buffer (2×) | 25 μL |
| T4 DNA ligase (1 U/μL) | 1 μL |
| Adapter (0.04 pmol/μL) | 5 μL |
| In total | 52 μL |

B. Incubating in a PCR instrument for 15 minutes at a temperature of 20° C.;

C. Purification—purifying the reaction products by magnetic beads, eluting with 19 μL of elution buffer EB (Elution Buffer) to obtain DNA fragments with adapters at both ends.

It should be noted that the fragments having A tailing to a single 3′ end obtained in (3) were subjected to blunt end ligation in this step.

(5) PCR amplification and purification of DNA fragments with both ends ligated to adapters

The DNA fragments having adapters added to both ends were subjected to PCR amplification to add base sequences matched with a sequencing primer to both ends of the DNA fragments in the process of PCR on the one hand, and to ensure that there is enough total amount of DNA fragments for the subsequent sequencing on the other hand.

Primer sequences used in PCR amplification:

PCR Primer 1:
5′-AATGATACGGCGACCACCGAGATCTACACTCTTTCCCTACACGA
CGCTCTTCCGATCT-3′
PCR Primer 2:
5′-CAAGCAGAAGACGGCATACGAGATCGTGATGTGACTGGAGTTCA
GACGTGTGCTCTTCCGATCT-3′

A. The reaction system is as follows:

| Reagent | Volume |
|---|---|
| DNA fragments having adapters added to both ends | 17 μL |
| 2 × DNA polymerase amplification mixture | 25 μL |
| PCR Primer 1 (10 pmol/μL) | 4 μL |
| PCR Primer 2 (10 pmol/μL) | 4 μL |
| In total | 50 μL |

B. After adding samples, placing in PCR and holding at 94° C. for 2 minutes; (94° C. for 15 seconds; 62° C. for 30 seconds; 72° C. for 30 seconds) 17 cycles; then 72° C. for 10 minutes; and finally storing the sample at 4° C.

C. Fragment selection and purification

The product obtained from the reaction was subjected to fragment selection and purification by magnetic beads (steps are described below) and was eluted with a certain amount of elution buffer EB (Elution Buffer), thereby completing the preparation of a DNA library for sequencing. The DNA library for sequencing was stored in a suitable environment for further use.

a. AmPure magnetic beads were equilibrated for 30 minutes at room temperature.

b. According to the number of samples to be purified, an equal number of 1.5 mL EP tubes were labeled as EP tubes.

c. The well-equilibrated magnetic beads of a were vortex blended, and 32.5 μL of magnetic beads were dispensed in the 1.5 mL EP tubes of b.

d. The PCR products and the magnetic beads of c were mixed and blended by mixing and rested at room temperature for 5 minutes. Then they were moved to the magnetic frame and stood for 3 minutes, and then the supernatant was discarded.

e. The 1.5 mL EP tubes of d were moved to the magnetic frame to stand for 3 minutes, and the supernatant was discarded.

f. 300 μL of 70% ethanol was added to the 1.5 mL EP tubes and the tubes were repeatedly mixed uniformity, and then the supernatant was discarded. This step was repeated. Then ethanol was absorbed clean and the tubes were allowed to dry at room temperature for 5 minutes.

g. The 1.5 mL EP tubes were moved from the magnetic frame to the EP tube holder and each EP tube was added with 52 μL of elution buffer and was mixed uniformity. The tubes were placed at room temperature for 5 minutes and then were moved to the magnetic frame standing for 1 minute.

h. The supernatant is the DNA library obtained after the fragment selection and purification. The supernatant was transferred to a new EP tube for direct sequencing or to store in a freezer of −20° C.

### Example 2

### Examining the Quality of the DNA Library for Sequencing

The fragment sizes of the DNA library constructed in Example 1 were tested using Agilent 2100 Bioanalyzer.

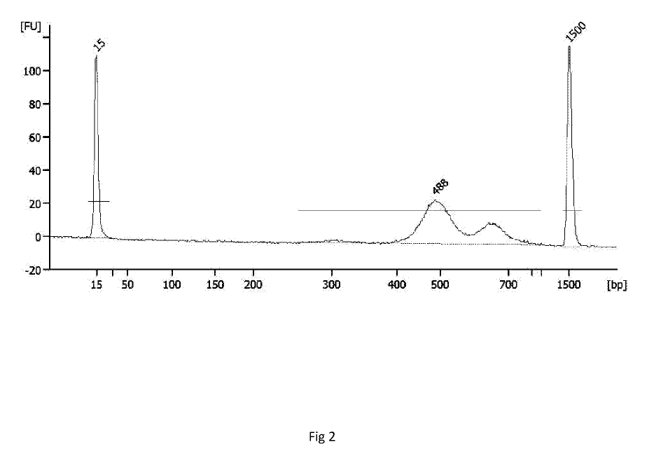

It is possible to know whether the constructed library fragments meet the above quality standards of the library by using Agilent 2100 Bioanalyzer to test the library. The test results are shown in FIG. 2. It can be seen from FIG. 2 that

(1) there are mainly two different sizes of fragments, 480 bp±10% and 660 bp±10%;

(2) the proportion of fragments of 480 bp±10% is about 50% to 70%, and the proportion of fragments of 660 bp±10% is about 30% to 50%.

This shows that the DNA library obtained in Example 1 is qualified.

Example 3

### Sequencing the DNA Library for Sequencing using Illumina NextSeq 500

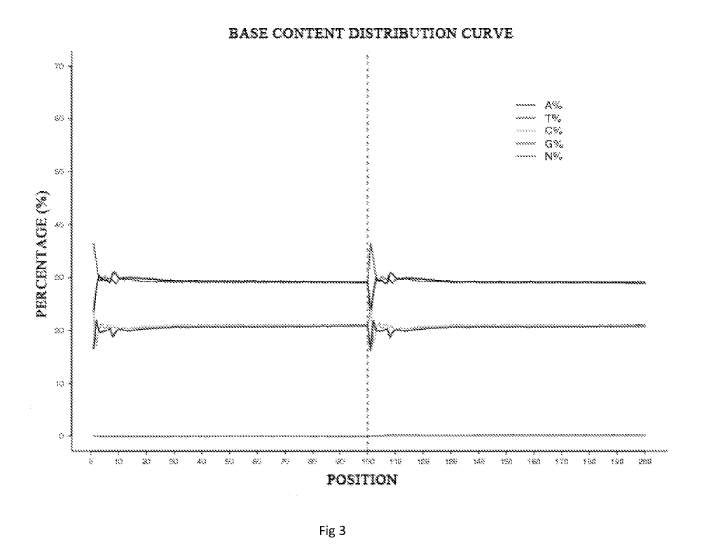

The DNA library for sequencing constructed in Example 1 was conducted by single-end sequencing and paired-end sequencing respectively, and the reagent used for sequencing was NextSeg™ 500 High Output v2 Kit. The sequencing results are shown in FIGS. 3 to 6.

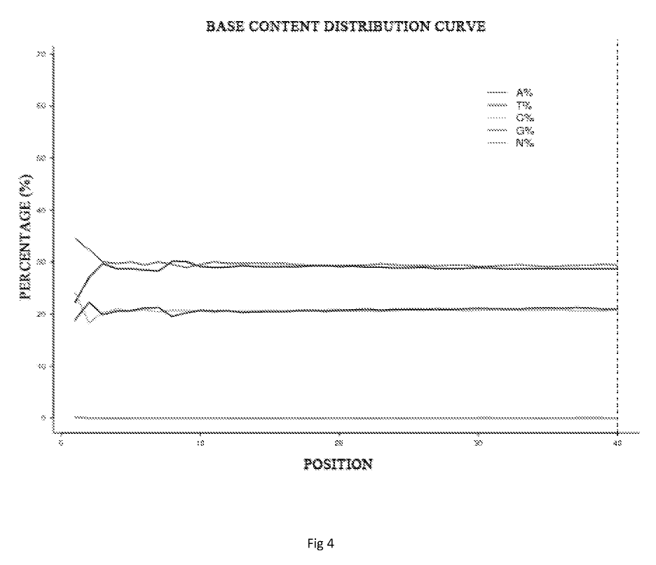

As can be seen from the base distribution diagrams of FIG. 3 and FIG. 4, the result of single-end sequencing of the above mentioned DNA library for sequencing and the result of paired-end sequencing of the above mentioned DNA library for sequencing are basically the same.

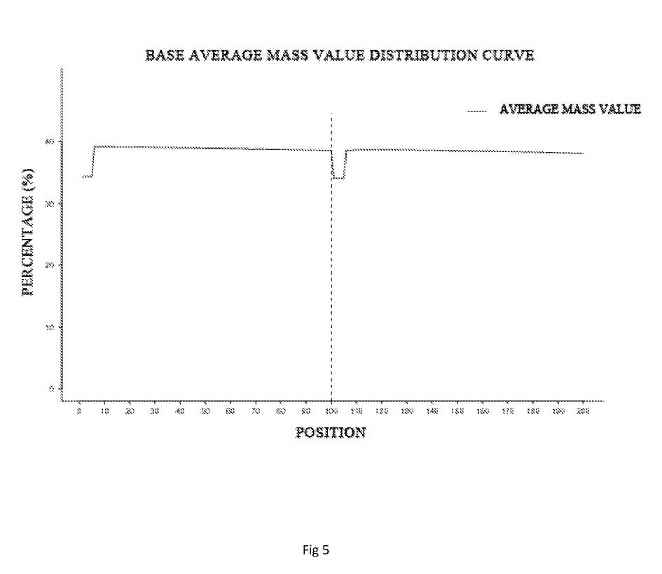

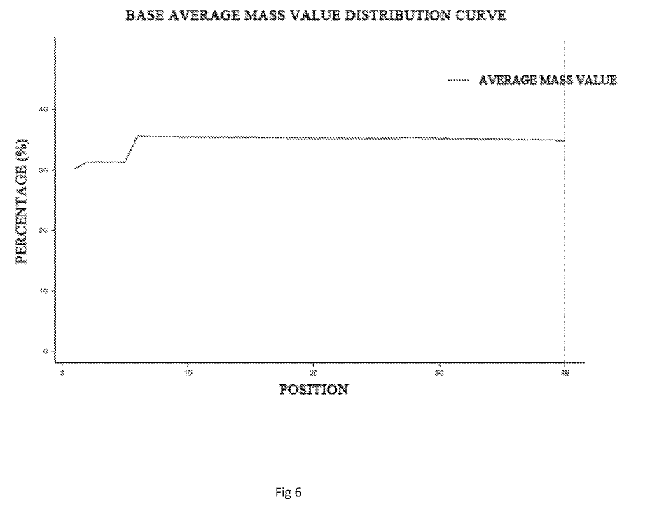

As can be seen from the mass distribution diagrams of FIG. 5 and FIG. 6, the mass curves are smooth and do not have falling points.

It should also be noted that any one of the technical features or combinations of the technical features described as constituents of a technical solution in the present specification may also be applied to other technical solutions on the premise that they can be practiced and do not contradict the gist of the present invention; moreover, the technical features described as constituents of different technical solutions may also be combined in any manner to form other technical solutions on the premise that they can be practiced and do not contradict the gist of the present invention. The present invention also includes technical solutions obtained by combinations in the aforementioned cases, and these technical solutions are regarded as being described in the present specification.

The above description shows and describes preferred examples of the present invention. As aforementioned, it should be understood that the present invention is not limited to the forms disclosed herein, and should not be construed as an exclusion of other examples, but may be applied to various other combinations, modifications and environments, and may be altered within the scope of the inventive concepts described herein by the above teachings or techniques or knowledge in related arts. Alterations and variations made by the skilled person in the art without departing from the spirit and the scope of the present invention are intended to be included within the scope of the appended claims of the present invention.

### INDUSTRIAL APPLICABILITY

The sequencing cost can be reduced and the sequencing efficiency can be improved in the sequencing using a DNA library for sequencing constructed by the method of constructing a DNA library for sequencing of the present invention.

1. A method for constructing a DNA library for sequencing, comprising:
step B-1: A-tailing of a single 3′ end to at least a portion of blunt-end DNA fragments to obtain DNA fragments having A tailing to the single 3′ end; and
step B-2: blunt-end ligating the DNA fragments having A tailing to the single 3′ end obtained in step B-1.

2. The method for constructing a DNA library for sequencing according to claim 1, further comprising:
step A performed before the step B: end-repairing the DNA fragments to be sequenced to obtain blunt-end DNA fragments;
step C performed after the step B-2 or simultaneously with the step B-2: adding adapters to the blunt-end ligated products of the step B-2 to obtain adapter-added DNA fragments; and
step D performed after the step C: subjecting the adapter-added DNA fragments to PCR amplification to obtain amplification products.

3. The method for constructing a DNA library for sequencing according to claim 2, further comprising: step E performed after the step D: fragment selection of the amplification products.

4. The method for constructing a DNA library for sequencing according to claim 3, further comprising: step F performed after the step E: purifying the amplification products after the fragment selection.

5. The method for constructing a DNA library for sequencing according to claim 2, further comprising:
step C1 performed after the step C: fragment selection of said adapter-added DNA fragments; and
in the step D, subjecting the adapter-added DNA fragments after the fragment selection to PCR amplification to obtain amplification products.

6. The method for constructing a DNA library for sequencing according to any one of claim 3, wherein amplification products containing two or more DNA fragments to be sequenced is selected.

7. A DNA library for sequencing, wherein double-stranded DNAs containing two or more DNA fragments to be sequenced makes up at least 10 mol % of the total double-stranded DNAs.

8. The DNA library for sequencing according to claim 7, which is constructed by the following method:
step B-1: A-tailing of a single 3′ end to at least a portion of blunt-end DNA fragments to obtain DNA fragments having A tailing to the single 3′ end; and
step B-2: blunt-end ligating the DNA fragments having A tailing to the single 3′ end obtained in step B-1.

9. A sequencing method, wherein sequencing is conducted using, a DNA library for sequencing, wherein double-stranded DNAs containing two or more DNA fragments to be sequenced makes up at least 10 mol % of the total double-stranded DNAs.

10. The sequencing method according to claim 9, wherein the sequencing is paired-end sequencing.

11. The sequencing method according to claim 9, wherein the sequencing is conducted using Illumina platform.

12. The DNA library for sequencing according to claim 7, which is constructed by the following method:
step A: end-repairing the DNA fragments to be sequenced to obtain blunt-end DNA fragments;
step B-1: A-tailing of a single 3′ end to at least a portion of blunt-end DNA fragments to obtain DNA fragments having A tailing to the single 3′ end;
step B-2: blunt-end ligating the DNA fragments having A tailing to the single 3′ end obtained in step B-1;
step C performed after the step B-2 or simultaneously with the step B-2: adding adapters to the blunt-end ligated products of the step B-2 to obtain adapter-added DNA fragments; and
step D: subjecting the adapter-added DNA fragments to PCR amplification to obtain amplification products.

13. The DNA library for sequencing according to claim 7, which is constructed by the following method:
step A: end-repairing the DNA fragments to be sequenced to obtain blunt-end DNA fragments;
step B-1: A-tailing of a single 3′ end to at least a portion of blunt-end DNA fragments to obtain DNA fragments having A tailing to the single 3′ end;
step B-2: blunt-end ligating the DNA fragments having A tailing to the single 3′ end obtained in step B-1;
step C performed after the step B-2 or simultaneously with the step B-2: adding adapters to the blunt-end ligated products of the step B-2 to obtain adapter-added DNA fragments;
step D: subjecting the adapter-added DNA fragments to PCR amplification to obtain amplification products; and
step E: fragment selection of the amplification products.

14. The DNA library for sequencing according to claim 7, which is constructed by the following method:
step A: end-repairing the DNA fragments to be sequenced to obtain blunt-end DNA fragments;
step B-1: A-tailing of a single 3′ end to at least a portion of blunt-end DNA fragments to obtain DNA fragments having A tailing to the single 3′ end;
step B-2: blunt-end ligating the DNA fragments having A tailing to the single 3′ end obtained in step B-1;
step C performed after the step B-2 or simultaneously with the step B-2: adding adapters to the blunt-end ligated products of the step B-2 to obtain adapter-added DNA fragments;
step D: subjecting the adapter-added DNA fragments to PCR amplification to obtain amplification products;
step E: fragment selection of the amplification products; and
step F: purifying the amplification products after the fragment selection.

15. The DNA library for sequencing according to claim 7, which is constructed by the following method:
step A: end-repairing the DNA fragments to be sequenced to obtain blunt-end DNA fragments;
step B-1: A-tailing of a single 3′ end to at least a portion of blunt-end DNA fragments to obtain DNA fragments having A tailing to the single 3′ end;
step B-2: blunt-end ligating the DNA fragments having A tailing to the single 3′ end obtained in step B-1;
step C performed after the step B-2 or simultaneously with the step B-2: adding adapters to the blunt-end ligated products of the step B-2 to obtain adapter-added DNA fragments;
step C1: fragment selection of said adapter-added DNA fragments; and
step D: subjecting the adapter-added DNA fragments after the fragment selection to PCR amplification to obtain amplification products.
